(19) United States
(12) Patent Application Publication
(10) Pub. No.: US 2003/0166995 A1
(43) Pub. Date: Sep. 4, 2003

(54) Method and device for mneasuring vital parameters

(76) Inventor: Klaus Jansen, Buxtehude (DE)

Correspondence Address: TECHNOPROP COLTON, L.L.C., P O BOX 567685, ATLANTA, GA, 311567685

(21) Appl. No.: 10/257,707

(22) PCT Filed: Apr. 12, 2001
(86) PCT No.: PCT/EP01/04237

(51) Int. Cl. A61B 5/00

(52) U.S. Cl. 600/300

(57) ABSTRACT

The invention relates to a method for measuring the vital parameters of a person, said vital parameters being measured and recorded by a non-invasive method. The vital parameters measured and/or data retrieved from a data processing of vital parameters are regularly recorded during the rest phases of the person on a long-term basis, that is over a period of days, weeks, months and/or years. The invention further relates to a corresponding device for measuring the vital parameters.

### BACKGROUND OF THE INVENTION

1. Technical Field

The invention relates to a method for measuring the vital parameters of a person, said vital parameters being measured and recorded by a non-invasive method. The invention further relates to a device for measuring the vital parameters of a person with at least one sensor for the non-invasive measurement of the vital parameters and with electronic memory for storing the measured vital parameters and/or the data acquired from data processing of the vital parameters.

2. Prior Art

The non-invasive measurement of vital parameters is already known, such as measuring heartbeat and respiration frequency, body temperature or electric body signals, for example, brain waves, by using the corresponding measuring devices such as a pulse measuring device or a thermometer. Also known are such devices capable of recording vital parameter measurement. One example is the so-called long-term ECG measuring device which is employed to measure electric cardiac signals over a period of many hours.

The known measuring methods present the disadvantage that their random sample character permits only a limited degree of significance.

### BRIEF SUMMARY OF THE INVENTION

The invention is therefore based on the objective of improving the acquisition of vital parameters.

This objective is solved by a method of the type stated above in that the measured vital parameters and/or data acquired from a data processing of vital parameters are regularly recorded during periods of rest, especially during sleeping phases, of the test person on a long-term basis, i.e. over a period of days, weeks, months and/or years. The objective is further solved by a device of the type stated above in that said device has a data storage configured such that the measured vital parameters and/or the acquired data can also be recorded and retrieved on a long-terms basis in the time periods outlined above.

The invention thus achieves a (long-term) biographic monitoring of vital parameters which can be advantageously employed in health care.

During periods of repose the body is generally in a stable state or constitution. In particular, no stress factors act upon the body. By measuring vital parameters during the repose period, one is able to avoid disturbing factors to a large degree. The parameters as measured by the invention are therefore very representative for the body's constitution.

Furthermore, the invention's measurement of vital parameters during phases of stabile body constitution is conducted over an extended period of time, namely over the course of days, weeks, months or years. This yields a large quantity of data on the vital parameters and such a large quantity provides an excellent base for statistical evaluation.

The measured vital parameters are preferably evaluated with respect to statistical variables, such as standard deviation, or variance. This evaluation is based on the knowledge that the circulation of a healthy person is characterized by relative high variances or standard deviations in cardiac frequency. On the other hand, a drop in variance or standard deviation indicates illness. For example, shortly before a myocardial infarction the variance approaches zero, i.e. the time interval between individual heart beats is essentially constant. The change in such statistic variables, such as variance or standard deviation, can enhance the knowledge concerning changes in the constitution of the person involved.

Of further preference the measured parameters or preprocessed vital parameters are subjected to a spectral transformation, i.e. transformed from the temporal range to the frequency range. This makes it possible to generate a frequency spectrum of the vital parameters or of the preprocessed vital parameters which allows one to conduct further studies on the attained spectrum or make conclusions about it. Further indicators concerning the physical constitution of a person can be in particular advantageously derived from such a spectral transformation and be made available for a further cycle of automation.

The stored or recorded data can be subjected to real-time data processing or bundled, for example, once daily or whenever additional conditions, such as clinical symptoms, emerge. The results of data processing are automatically generated in a report. This report can be generated either by a local data processing installation, i.e. near the test person being measured, or by a central databank. The device according to the invention prefers a data transfer installation for remote data transmission which can not only send data from the local measuring device to the central databank but can also receive data from the central databank.

The central databank archives and governs the measured vital parameters, or the variables derived therefrom, of a plurality of persons. This data exchange allows for central data management concerning many persons, in particular with the knowledge obtained from the statistical evaluation of the data on many persons also benefiting other persons, for example by determining further, generally valid reference variables on the strength of a broad-based databank compiled from the data on a plurality of persons. In particular, not only are the data on a single person evaluated among one another, but are also set against the data of other persons. This allows one to reach new findings concerning pathological and non-pathological vital parameters or variables derived from the vital parameters. Furthermore, the data transmission device can also be used advantageously for software maintenance, i.e. for updating the software of the local measuring installation.

### BRIEF DESCRIPTION OF THE DRAWINGS

Further details and special features of the invention will be illustrated more closely in the following as based on an exemplary embodiment represented in the drawing, which shows:

FIG. 1 the principal structure of a mattress in side view with a measuring device;

FIG. 2 a block diagram illustrating the data processing of the measured vital parameters.

### DETAILED DESCRIPTION OF THE PREFERRED EMBODIMENTS

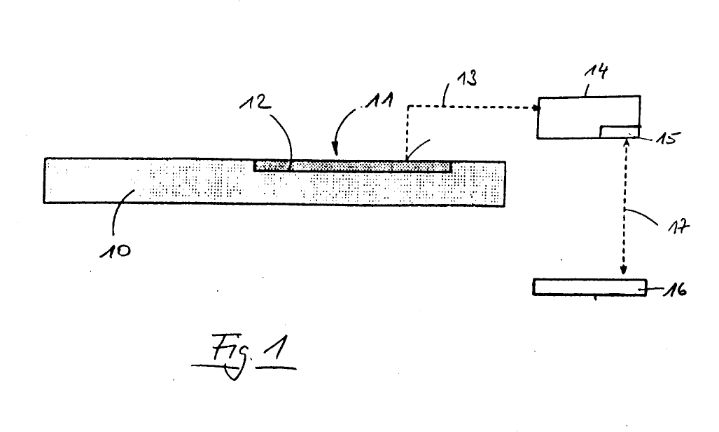

FIG. 1 shows a mattress 10 which has on its top side 11 a recess for an insert 12 having one or more sensors. These sensors are pressure and/or temperature sensors, acoustic sensors, such as microphones, and/or electrodes. These sensors are arranged in the insert 12 such that it is possible to measure the vital parameters of the person lying on the mattress 10 in a non-invasive manner. The sensors are connected via a measurement connection 13 to a data processing unit 14. The measured vital parameters are processed in said data processing unit. Furthermore, the data processing unit 14 has a data transmission device 15, by means of which the measured vital parameters and/or the data obtained from data processing can be transmitted to a central databank 16. To this end, the data transmission device is advantageously equipped with a modem, by means of which it is possible to establish a data link to the central databank 16 via a data network, or a remote data communications network 17, or a telephone line, and vice versa.

The data processing unit 14 and/or the central databank 16 has memory storage for the measured vital parameters and/or the obtained data which is configured, or organized, such that data can be recorded and retrieved on a long-term basis, i.e. over a period of days, weeks, months and/or years. To this end the data are in particular linked to additional specifications, such as date and time of day, so that a temporal association can also be made for data on different vital parameters. This requires that the memory storage has a sufficiently large capacity for managing the corresponding amount of data.

Furthermore, the data processing unit 14 and/or central databank 16 has one or more computers or microprocessors capable of conducting statistical calculations such as mean value, standard deviation, or variance, and also capable of conducting spectral transformations. In particular, the data processing unit 14 or the central databank 16 has what is known as a FFT analyzer, i.e. means for conducting a Fourier transformation, preferably a discrete Fourier transformation, such as the so-called fast Fourier transformation (FFT), or other devices for executing another spectral transformation. For example, such a device could be used to conduct spectral transformations such as the so-called Laplace transformation or the Hilbert transformation, or other transformations based on complete functions.

The data processing unit 14 also has a operating unit which, for example, can be placed on a bedside table. In an advantageous development, this operating unit is connected to an alarm clock with is generally present on the bedside table in any case. This is especially advantageous in that the measured or calculated data can be stored along with the corresponding date and time of day.

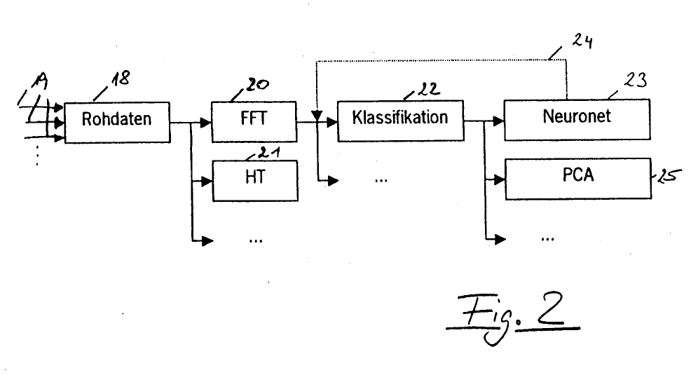

FIG. 2 shows a block diagram of data processing as implemented. In a first block 18 the vital parameters are measured or collected. To this end a plurality of input ports 19 are provided. In these input ports 19 measurements are taken by sensors contained in the insert 12, in particular measurements concerning pressure, temperature and/or acoustic and/or electric signal levels. The raw data collected in this manner from the first block 18 are then subjected to further data processing in a second block 20, third block 21 or a further block. The second block 20 is configured as a block for implementing a Fourier transformation and the third block for implementing a Hilbert transformation. The signals collected by the first block 18 can thus be depicted in a state space based on complete functions. A fourth block 22 executes a classification of an output signal of the second, third or a further block, preferably with the assistance of neuronal network algorithms as represented by the fifth block 23 as well as the feedback 24 from the fifth block 23 to the fourth block 22. The result of the classification of the fourth block 22 is further processed in a sixth block 25 by means of a so-called primary component analysis.

The data processed in this manner, or the results of data processing, can be regularly retrieved by the attending person in the form of a machine-generated report. This provides one with information about the constitution of the observed person, but also in particular about any trends, such as an improvement or deterioration of the body's condition. Essential for this purpose is an extensive databank or data pool that collects data over extended periods of time and evaluates them statistically. A central databank or central data pool also offers the advantage that data can be evaluated by third parties from a remote location. In addition, by means of said data transmission it is possible to update and maintain the software running in the data processing unit 14 as soon as the broad data base leads to improved data processing algorithms.

The described method and device have the advantage that the constitution of a person can be monitored continuously and automatically over an extended period of time without impairing or detracting the person involved. In particular the measurement of vital parameters during phases of repose, especially at night, means that measurement does not take up any of the person's time at all. In addition, the person under observation does not feel the measurement being taken since it is conducted in a non-invasive manner, in particular with no direct contact to the sensor or sensors involved.

Furthermore the described device offers the possibility of generating an alarm signal when a limit value, either pre-determined or obtained from the data processing, is met or exceeded. This alarm signal indicates to the person under observation that his or her state of health has deteriorated. This monitoring and alarm process can be carried out fully automatically without the interim assistance of a physician or medical personnel. Instead, the generation of an alarm is based on the statistical evaluation of the measured vital parameters, in particular the standard deviation, or variance, in the heart rate. This means that when this standard deviation, or variance, drops below a certain limit, an alarm signal is generated by the data processing unit 14 or central databank 16. Preferably, however, an alarm is not generated if a limit value is exceeded for only a brief interval. Rather, the sounding of an alarm also takes the biographical context into account, i.e. a trend is examined and followed which allows inferences to be made concerning steps to be taken for treatment or for making a specific diagnosis.

On the whole, the invention's combination of non-invasive measurement of vital parameters and their long-term recording permits additional evaluation which yields information about the constitution of the person under observation. To this end, the measured variables are in particular subjected to a statistical evaluation, such as the calculation of a standard deviation, or variance from a calculated mean value. In addition, the discussed spectral transformation of the measured or processed vital parameters allows additional information to be gained from the data obtained. On the whole, by virtue of the invention, the health care provided is considerably improved through the biographically-based collection of vital parameters.

LIST OF DESIGNATIONS

| | |
|---|---|
| 10 | mattress |
| 11 | topside |
| 12 | insert |
| 13 | measurement connection |
| 14 | data processing unit |
| 15 | data transmission device |
| 16 | central databank |
| 17 | remote data communications network |
| 18 | first block |
| 19 | input port |
| 20 | second block |
| 21 | third block |
| 22 | fourth block |
| 23 | fifth block |
| 24 | feedback |
| 25 | sixth block |

1. Method for measuring the vital parameters of a person, said vital parameters being measured (18) and recorded in a non-invasive manner, characterized in that the measured vital parameters and/or data acquired from a data processing of vital parameters are regularly recorded during periods of rest, especially during sleeping phases, of the person on a long-term basis.

2. Method according to claim 1, characterized in that respiration frequency, heartbeat, body temperature and/or electric body signals are measured as vital parameters.

3. Method according to claim 2, characterized in that the data processing includes a spectral transformation selected from the group consisting of Fourier transformation (20), Hilbert transformation (21), Laplace transformation, or a transformation based on complete functions.

4. Method according to claim 3, characterized in that the data processing includes a classification (22) of the data obtained from the transformation.

5. Method according to claim 4, characterized in that the data processing includes a calculation of the standard deviation, or variance, of a vital parameter.

6. Method according to claim 5, characterized in that an alarm signal is generated when a pre-determined limit value or a limit value obtained from the data processing is met or exceeded.

7. Method according to claim 6, characterized in that the measured vital parameters and/or obtained data are transmitted to a central databank (16), with the databank (16) archiving the data of a plurality of persons.

8. Method according to claim 7, characterized in that the central databank (16) processes the archived data of one or more persons with or among each other.

9. Device for measuring the vital parameters of a person with at least one sensor (12) for the non-invasive measurement of the vital parameters and a memory storage for storing the measured vital parameters and/or the data obtained from a data processing of the vital parameters, characterized in that the memory storage is configured such that the measured vital parameters and/or the obtained data can be recorded over an extended period of time and can be retrieved at need.

10. Device according to claim 9, characterized in that the sensor or sensors (12) are arranged on and/or in a mattress (10).

11. Device according to claim 10, characterized by a data processing unit (14) for processing the measured vital parameters.

12. Device according to claim 11, characterized by a data transmission device (15) for the transmission of measured vital parameters or of data obtained from the data processing to a central databank (16) and/or for the reception of data.

13. Device according to claim 12, characterized in that the sensor (12) has a sensor selected from the group consisting of temperature sensors, pressure sensors, and acoustic sensors.

14. Device according to claim 13, characterized in that the data processing unit can be placed in the region of the mattress (10) and has an alarm clock.

15. Device according to claim 9 for measuring the vital parameters of a person with at least one sensor (12) for the non-invasive measurement of the vital parameters and a memory storage for storing the measured vital parameters and/or the data obtained from a data processing of the vital parameters, characterized in that the memory storage is configured such that the measured vital parameters and/or the obtained data can be recorded over an extended period of time and can be retrieved at need, wherein the measured vital parameters and/or data acquired from a data processing of vital parameters are regularly recorded during periods of rest, especially during sleeping phases, of the person on a long-term basis.

16. Method according to claim 5, wherein the vital parameter is a heartbeat.

17. Method according to claim 6, wherein the alarm signal is generated when the standard deviation or variance of the heartbeat exceeds a limiting value.

18. Device according to claim 12, wherein the data is selected from the group consisting of an alarm signal and software updates provided by a central databank.

19. Device according to claim 13, wherein the sensor is a microphone.

20. Device according to claim 14, wherein the device can be placed on a bedside table associated with the mattress.
